(19) United States
(12) Patent Application Publication
(10) Pub. No.: US 2015/0258248 A1
(43) Pub. Date: Sep. 17, 2015

(54) MEDICAL TUBES COMPRISING COPPER-BASED COMPOUND

(71) Applicant: BS SUPPORT CO., LTD., Suwon-si (KR)

(72) Inventor: Seung Woo BAEK, Anyang-si (KR)

(21) Appl. No.: 14/705,246

(22) Filed: May 6, 2015

Related U.S. Application Data
(63) Continuation-in-part of application No. PCT/KR2014/010938, filed on Nov. 14, 2014.

(30) Foreign Application Priority Data
Mar. 17, 2014 (KR) 10-2014-0030733

(51) Int. Cl. A61L 29/10; A61L 31/16; A61L 31/04; A61L 31/08; A61L 29/04; A61L 29/16

(52) U.S. Cl. CPC A61L 29/106; A61L 29/04; A61L 29/16; A61L 31/04; A61L 31/088; A61L 31/16; A61L 2300/102

(57) ABSTRACT

A medical tube comprising a copper-based compound, which is relatively inexpensive, is easy to process, is not toxic, and has excellent antibacterial activity, comprises: a tube comprising a polymer resin and having a predetermined shape and diameter; and a copper-based compound coated on the surface of the tube or dispersed in the polymer resin of the tube, wherein the compound has a chemical structure of CuₓM_{y}, wherein M is any one selected from groups 15 to 17 of the periodic table, and x/y is 0.5-1.5.

### CROSS REFERENCE TO RELATED APPLICATIONS

This application is a continuation-in-part application of International Application No. PCT/KR2014/010938, filed on Nov. 14, 2014, which claims priority to and the benefit of Korean Application No. 10-2014-0030733, filed on Mar. 17, 2014, in the Korean Intellectual Property Office, the entire contents of which are incorporated herein by reference.

### BACKGROUND OF THE INVENTION

1. Field of the Invention

The present invention relates to a medical tube comprising a copper-based compound, and more particularly, to a medical tube comprising an electrically conductive copper-based compound that improves the antibacterial activity of the medical tube.

2. Description of the Prior Art

Medical tubes include tubes for injecting drugs, biological fluids or the like into the body or extracting them from the body, catheters that are inserted into the body to perform examination, treatment or the like, etc. Specifically, medical tubes include tubes for infusion, entreat nutrition, peritoneal dialysis, transfusion, or transfer of urine into a urine collection bag, tubes for use in blood circuits for blood dialysis, blood circuits for artificial heart lung machines, or blood circuits for plasma exchange, tubes for mass transfer in the medical field, etc. The tubes for mass transfer include, for example, tubes attached to multiple blood bags, tubes that are used to connect catheters to suction units, etc. In addition, catheters include urinary catheters, gavage catheters, suction catheters, etc.

Meanwhile, pathogenic bacteria are easily colonized on the surface of medical tubes. Medical tubes having pathogenic bacteria colonized thereon may cause serious contamination problems. In the prior art, silver (Ag) and silver ions, which release silver ions, have been used to prevent the colonization of pathogenic bacteria. Silver (Ag) is highly toxic to bacteria even at a very low concentration, and pathogenic bacteria are less likely to develop resistance to silver. U.S. Pat. No. 3,800,087 discloses a catheter having silver coated on the outer wall thereof. However, in the patent document, the adhesion of silver to the surface is poor.

In an attempt to increase the adhesion of silver, German Patent No. 4328999 discloses applying a metal layer having a better adhesive property between a plastic material and a silver coating. However, applying the metal layer requires a very complex process and is costly, and the amount of silver ions that are used for antibacterial purposes is insignificant compared to the amount of the applied silver. In addition, it is difficult to form a silver coating on the inner surface of a tube by application.

To overcome the above-mentioned problems, salts of silver (Ag) have been used in antibacterial coatings in some cases. However, unlike silver, salts of silver can have anions that can be toxic in a particular environment. In addition, some silver salts such as silver nitrate are highly soluble in water, and thus when they are coated on surfaces, silver ions can be transferred to the surrounding environment in a too early stage. Further, other silver salts such as silver chloride have poor solubility in water, and thus silver ions can be transferred from the silver solution in a too late stage. There are various known methods for incorporating nanocrystalline silver into a plastic material. These methods for incorporating nanocrystalline silver into a plastic material are described, for example, in WO 01/09229A1, WO 2004/024205 A1, EP 0 711 113 A, and Muenstedt et al., Advanced Engineering Materials 2000, 2(6), pages 380-386. However, the methods described in these published documents have disadvantages in that the amount of silver remaining on polyurethane pellets after dipping is not constant and cannot be previously determined.

Korean Patent No. 10-0987728 discloses producing an antimicrobial yarn by depositing silver on a resin surface using a sputtering or ion-plating method and adding the deposited silver. Korean Patent No. 10-1180117 discloses producing an antimicrobial yarn by adsorbing zinc sulfide nanoparticles and an organic antimicrobial agent. Although the silver and sulfur components used in the above prior art documents have high antibacterial activity, there are many limits to the practical use thereof. Specifically, silver has high antibacterial activity and convenience, but is excessively costly. Sulfur has problems in that it is environmentally toxic and is difficult to process, and these problems have not yet been solved.

### SUMMARY OF THE INVENTION

It is an object of the present invention to provide a medical tube comprising a copper-based compound, which is relatively inexpensive, is easy to process, is not toxic, and has excellent antibacterial activity.

To accomplish the above object, the present invention provides a medical tube comprising: a tube formed of a polymer resin and having a predetermined shape and diameter; and a copper-based compound coated on the surface of the tube or dispersed in the polymer resin. Herein, the compound has a chemical structure of CuₓM_{y}, wherein M is any one selected from groups 15 to 17 of the periodic table, and x/y is 0.5-1.5.

In the medical tube of the present invention, M in the chemical formula may be any one selected from among S, F and Cl, and the compound is preferably copper sulfide. Moreover, the tube having the compound dispersed in the polymer resin comprises, based on the total weight of the medical tube, 0.1-5 wt % of metal particles of at least one selected from among chromium, manganese, iron, cobalt, nickel and zinc. Herein, the average particle size of the metal particles is preferably smaller than the average particle size of the compound.

Coating of the compound on the surface of the tube may be performed by any one method selected from among wet coating, vapor deposition, and plating. Before the compound is coated on the tube, a coating solution containing 0.01-3.0 wt % of colloidal transition metal particles and 0.01-5.0 wt % of at least one emulsion selected from among water-soluble polyester, water-soluble urethane and water-soluble acryl may be applied to the medical tube.

In a preferred embodiment of the present invention, the medical tube is any one selected from among tubes for infusion, enteral nutrition, peritoneal dialysis, transfusion, or transfer of urine into a urine collection bag, tubes for use in blood circuits for blood dialysis, blood circuits for artificial heart lung machines, or blood circuits for plasma exchange, tubes for endoscopy, tubes for mass transfer in the medical field, and catheters. The tube for mass transfer may be a tube attached to a multiple blood bag, or a tube that is used to connect a suction unit to a catheter. The catheters may include a urinary catheter, a gavage catheter or a suction catheter. The medical tube of the present invention may be composed of a plurality of tubes connected by a connector, such as a plurality of catheters connected by a connector.

### BRIEF DESCRIPTION OF THE DRAWINGS

FIG. 1 is a photograph showing copper sulfide particles prepared in an Example of the present invention.

FIG. 2 is a XRD graph showing the crystalline structure of copper sulfide prepared in an Example of the present invention.

FIG. 3 is a micrograph (30,000x) of copper sulfide prepared in an Example of the present invention.

### DETAILED DESCRIPTION OF THE INVENTION

Hereinafter, preferred embodiments of the present invention will be described in detail with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art.

Embodiments of the present invention provide a medical tube comprising a copper sulfide-containing compound, which is relatively inexpensive, is easy to process, is not toxic, and has high antibacterial activity. For this purpose, the medical tube comprising the composition dispersed or coated thereon will be specifically examined, and the antibacterial activity of the medical tube will be specifically examined. Meanwhile, the medical tube according to the present invention may be produced either by coating a compound on the surface of a tube by deposition or adsorption or by compounding particles of the compound with a polymer resin.

The medical tube of the present invention may be produced by processing a tube having a specific diameter into a desired shape, and a functional part such as a hole may, if necessary, be formed in the tube. Examples of the medical tube include tubes for injecting drugs, biological fluids or the like into the body or extracting them from the body, catheters that are inserted into the body to perform examination, treatment or the like, etc. Specifically, medical tubes include tubes for infusion, enteral nutrition, peritoneal dialysis, transfusion, or transfer of urine into a urine collection bag, tubes for use in blood circuits for blood dialysis, blood circuits for artificial heart lung machines, or blood circuits for plasma exchange, tubes for mass transfer in the medical field, etc. The tubes for mass transfer include, for example, tubes attached to multiple blood bags, tubes that are used to connect catheters to suction units, etc. The medical tube of the present invention may be composed of a plurality of tubes connected by a connector, such as a plurality of catheters connected by a connector.

The medical tube may be made of polymer resin such as thermoplastic resin or thermosetting resin. Preferably, the medical tube is made of thermoplastic resin which is easy to mold. Major examples of the thermoplastic resin include polyethylene terephthalate, polylactic acid, polyethylene, polypropylene, polycarbonate, polymethylmethacrylate, polyvinyl chloride, silicone, etc. The thermosetting resin is preferably epoxy resin or the like. Meanwhile, polyvinyl chloride (PVC) has been widely used to date for medical tubes due to its excellent processability and convenience, but the use thereof has gradually decreased, because environmental regulations on the emission of toxic substances have become more stringent in recent years. Rather, olefinic resins have been increasingly used, such as low-density polyethylene (LDPE), high-density polyethylene (HDPE) or polypropylene (PP). In recent years, polylactic acid (PLA) that is a biomaterial produced from corn or potatoes has also been used. Polyurethane is more preferably used, because it is flexible and non-toxic and has good chemical resistance.

The copper-based compound that is used in the embodiment of the present invention is preferably copper sulfide (CuS). In the present invention, copper sulfide was synthesized by reacting copper sulfate (CuSO₄) with a sulfide salt, at a molar ratio of 1:1 in an aqueous solution at a temperature of 10 to 80° C. Herein, the synthesized copper sulfide had a chemical formula of CuₓS_{y}, and the synthesis conditions were set such that x/y in the chemical formula would satisfy 0.5-1.5. Examples of a sulfide salt that may be used in the present invention include sodium sulfide, iron sulfide, potassium sulfide, zinc sulfide, etc, In the present invention, copper sulfide synthesized by reacting copper sulfate with sodium sulfide had the highest antibacterial activity.

Meanwhile, if the reaction temperature is lower than 10° C., the resulting copper-based particles will have good antibacterial activity, but the reactivity between copper sulfate and a salt during the synthesis of the particles, and the yield of production of copper sulfide will be low. If the reaction temperature is higher than 80° C., the reaction rate will be excessively high, the crystalline density of the surface of the resulting copper sulfide will increase, and concentration of copper will increase to reduce the antibacterial activity of the resulting copper sulfide. In addition, if the x/y ratio of the copper-based particles is lower than 0.5, the concentration of sulfur (S) will excessively increase to increase the antibacterial activity, but the chemical stability of the resulting copper sulfide will decrease. If the x/y ratio of the copper-based particles is higher than 1.5, the concentration of copper will increase to reduce the antibacterial activity.

Hereinafter, a process for producing a medical filter will be described, which is divided into a process of coating the compound copper sulfide on a medical tube, and a process of dispersing copper sulfide particles on a medical tube.

Medical Tube Coated with Copper Sulfide

Coating the surface of a medical tube with copper sulfide according to an embodiment of the present invention may be performed by various processes, including wet coating, plating and deposition. The wet-coating process has advantages in that it is simple or inexpensive, even though it shows low adhesive strength compared to the plating or deposition process. In the coating process, 1-30 wt % of copper sulfide powder is added to and sufficiently dispersed in a solvent containing at least one of IPA, toluene, benzene, a binder and the like, and the dispersion may be coated on a medical tube by a method such as dip coating, spray coating or the like. The concentration of copper sulfide is determined by taking into consideration the dispersibility and thickening thereof. When a dispersing agent is used, a high-concentration coating solution can be prepared.

Copper sulfide is preferably coated on the medical tube to a thickness of about 300-600 Å, and the coating thickness can be controlled by repeating the coating process or controlling the viscosity of the coating solution. The coated tube is dried. Preferably, the coated tube is subjected to a low-temperature drying step, followed by a sintering step. The drying step is a step of slowly removing water and the solvent from the coated tube, and is preferably carried out at a temperature of 90 to 100° C. for 1-hours. The sintering step is a step of increasing the binding strength between copper sulfides. Because copper sulfide is likely to be decomposed at 400° C., the sintering step is preferably carried out at a temperature of 200 to 300° C. for 1-2 hours. If the drying step is carried out at an excessively high temperature for an excessively long time, the coating layer will be cracked to deteriorate the appearance, and sulfur will be separated from the coating layer, resulting in a significant decrease in the antibacterial activity of the coating layer. Particularly in the case of spray coating, a coating solution prepared using a supercritical fluid such as carbon dioxide is more preferably used. The supercritical fluid can overcome the toxic problem of organic solvents, and makes it possible to reduce the drying time.

In the deposition process, copper sulfide having a chemical formula of CuₓM_{y} (M is any one selected from among S, F and Cl, and x/y=0.5-1.5) is synthesized, which is to be vacuum-deposited. To the surface of a tube, an aqueous coating solution containing 0.01-3.0 wt % of colloidal transition metal particles and 0.01-5.0 wt % of at least one emulsion selected from among water-soluble polyester, water-soluble urethane and water-soluble acryl is applied. The aqueous coating solution is controlled such that it leaves solids in an amount of 0.001-0.1 g/m². In the deposition process, heating is performed under a vacuum of 10⁻⁵-10⁻³ Torrso that the vapor pressure of the metal is maintained at 10⁻²-10⁻¹ Torr, thereby depositing copper sulfide on the surface of the tube to a thickness of 300-600 Å. The deposited layer preferably has an adhesive strength of at least 60 g/25 mm.

The plating process provides a tube that has high durability so as to be suitable for repeated use for a long period of time, even though it has disadvantages in that it is difficult to carry out and is expensive, compared to the deposition or wet-coating process. To increase the adhesive strength of the plated layer, a process of treating the tube surface with an electrically conductive polymer emulsion containing a transition metal is performed before the plating process. To the tube surface, an aqueous coating solution containing 0.01-1.0 wt % of colloidal transition metal particles and 0.01-2.0 wt % of at least one emulsion selected from among water-soluble polyester, water-soluble urethane and water-soluble acryl is applied. The aqueous coating solution is controlled such that it leaves solids in an amount of 0.001-0.1 g/m². The plating process may also be performed by ionizing copper sulfide in a solvent and applying the ionized solution to the tube surface by electroplating or electroless plating. For example, the plating process may be performed by adding a copper salt and a sulfur-containing compound to a plating solution and precipitating copper sulfide by a reducing agent. Preferably, copper sulfide is plated on the tube to a thickness of 0.01-5.0 μm.

Among the above-described processes for coating copper sulfide on the tube surface, the dip-coating process was used in an Example of the present invention. Specifically, a predetermined amount of copper sulfide was added to a solvent such as isopropyl alcohol (IPA) and stirred at room temperature for several hours to prepare a coating solution having good dispersibility. Then, the medical tube was dip-coated with the coating solution. The coated medical tube was dried at a temperature of a few tens of ° C., and then annealed for several minutes at a temperature between the crystallization temperature (T_{c}) and melting temperature of the polymer resin forming the medical tube. To impart excellent antibacterial activity to the medical tube, the coating process was repeated so that copper sulfide can be coated on the medical tube surface to a sufficient concentration.

Medical Tube Having Copper Sulfide Particles Dispersed Therein

The medical tube according to the embodiment of the present invention is preferably composed of a mixture of the polymer resin and greater than 0 wt % but smaller than 50 wt % of copper sulfide particles. Herein, the sulfur content of the synthesized copper sulfide particles is preferably 40-60 mole %. If the sulfur content of the particles is less than 40 mole %, the antibacterial activity of the particles will have poor antibacterial activity, and if the sulfur content is more than 60 mole %, it will be difficult to synthesize copper sulfide. However, when the copper sulfide particles according to the embodiment of the present invention is compounded with the polymer resin in order to produce a medical tube, the dispersibility of the copper sulfide particles will be reduced. For this reason, the pressure in the process of extruding the compounded material (extrusion pressure) may increase. In order to prevent the extrusion pressure from increasing, according to an embodiment of the present invention, metal particles of at least one transition metal selected from chromium, manganese, iron, cobalt, nickel and zinc, which belong to group 4 of the periodic table, may be added to the tube in an amount of 0.1-5 wt % based on the total weight of the tube. If the transition metal is mixed with the copper-based compound, the mixture will have excellent dispersibility and antibacterial activity, compared to a typical metal such as aluminum (Al).

Meanwhile, the average particle size of the metal particles is preferably smaller than the average particle size of the copper-based compound particles. In addition, if the amount of metal particles added when compounding copper sulfide with thermoplastic resin is more than 0.1 wt % or less than 5 wt %, the extrusion pressure may decrease rather than increase. As described above, the metal particles are added in order to control the extrusion pressure, and antibacterial activity required for the medical tube can be obtained even only by the copper-based compound. Thus, producing the medical tube without using the transition metal particles also fall within the scope of the present invention. Herein, transition metal particles that are added to the medical tube of the present invention are selected from those that do not impair the antibacterial activity of the medical tube.

In an Example of the present invention, compounding was used to increase the dispersibility of the particles in the polymer resin, and the compounding was performed at a barrel temperature that was 30 to 50° C. higher than the melting temperature of the polymer resin. The compounding was performed in a compounding machine equipped with a biaxial unidirectional screw having excellent dispersibility compared to a monoaxial screw. The compounding machine preferably a length (L)/diameter (D) ratio ranging from 30 to 40. The compounded resin was stored in the form of chips in a bunker, and then extruded at a temperature that was 30 to 50° C. higher than the melting temperature of the polymer resin used. Next, the extruded resin was subjected to molding, first-step cooling, annealing and second-step cooling, thereby producing a medical tube of the present invention.

Hereinafter, the present invention will be described in further detail with reference to the following examples. It is to be understood, however, that these examples are for illustrative purposes only and are not intended to limit the scope of the present invention. The performance of tubes produced in Examples of the present invention and Comparative Examples was evaluated in the following manner.

(1) Antibacterial Activity

To evaluate the antibacterial activity of each test specimen, Escherichia coli (ATCC 25922) used as a test bacterial strain was brought into contact with each test specimen, and then stationarily cultured at 25° C. for 24 hours, after which the number of the bacterial cells was counted.

(2) Extrusion Pressure

The dispersibility of copper sulfide and metal particles in polymer resin was evaluated based on a change in extrusion pressure applied to a filter. Specifically, a change in filter pressure (4P) applied to a 350-mesh filter when extruding 30 kg/hr of resin through a pilot extruder was measured. As the change in the filter pressure was lower, the dispersibility of copper sulfide and metal particles was evaluated to be better.

### Example 1

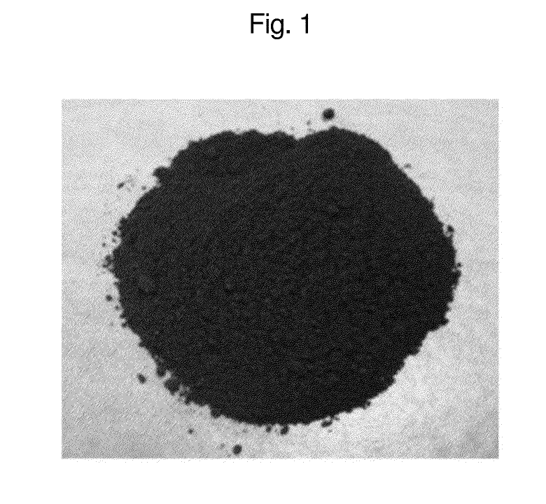

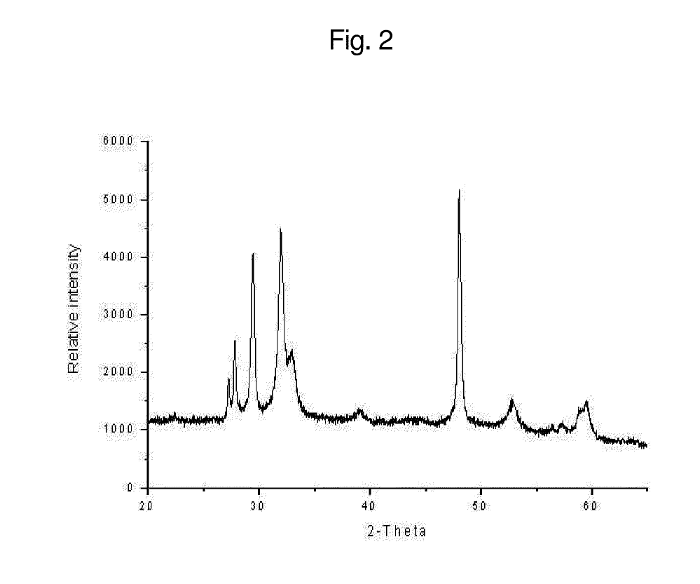

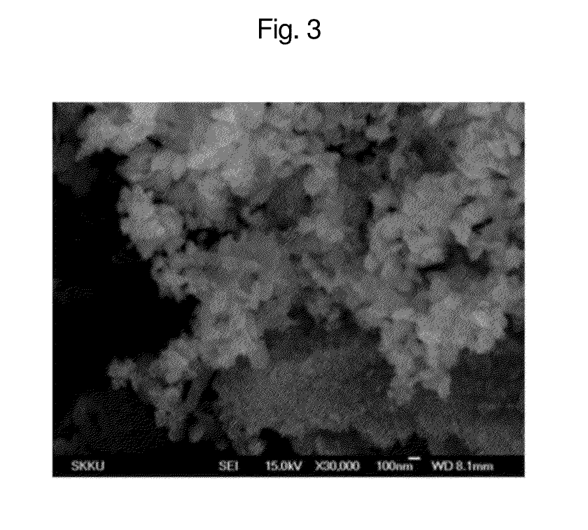

1 mole of each of CuSO₄ and Na₂S was added to distilled water and stirred for 30 minutes. Then, the stirred solution was introduced into an isothermal reactor at 50° C. and allowed to react for 30 minutes, thereby synthesizing copper sulfide particles as shown in FIG. 1. The synthesized copper sulfide had the characteristic crystalline structure of copper sulfide as shown in FIG. 2, and the morphology of the particles observed at 30,000× magnification is as shown in FIG. 3. As shown in FIG. 2, the peak of sulfur did not appear because sulfur has no crystalline structure, but the peak of copper appeared at 55, 65, 99, 125 and 137 degrees. Observation of the particles was performed by X-ray powder diffraction (XRD, XD-3A, Shimadzu, Japan).

In a process of coating the surface of a medical tube with the copper sulfide synthesized as described above, 5 wt % of the copper sulfide was added to isopropyl alcohol (IPA) and stirred at room temperature for 1 hour to thereby prepare a coating solution having excellent dispersibility. The coating solution was dip-coated on a medical tube having a diameter of 1 cm and a length of 10 cm. The coated tube was first dried at 50° C. for 1 hour, and then annealed for 30 minutes at a temperature between the crystallization temperature (T_{c}) and melting temperature of the polymer resin forming the medical tube. The coating process was repeated in the same manner as described above so that copper sulfide could be coated on the surface of the medical tube to a sufficient concentration, thereby providing a medical tube having excellent antibacterial activity. The antibacterial activity of the tube prepared as described above was measured according to the above-described method.

### Example 2

A coating solution containing 1 wt % of copper sulfide synthesized as described in Example 1 was dip-coated on a medical tube made of low-density polyethylene (LDPE; specific gravity: 0.92) and having a diameter of 1 cm and a length of 10 cm. The antibacterial activity of the tube prepared in this Example was measured according to the above-described method.

### Example 3

A coating solution containing 10 wt % of copper sulfide synthesized as described in Example 1 was dip-coated on a medical tube made of low-density polyethylene (LDPE; specific gravity: 0.92) and having a diameter of 1 cm and a length of 10 cm. The antibacterial activity of the tube prepared in this Example was measured according to the above-described method.

### Example 4

A coating solution containing 30 wt % of copper sulfide synthesized as described in Example 1 was dip-coated on a medical tube made of low-density polyethylene (LDPE; specific gravity: 0.92) and having a diameter of 1 cm and a length of 10 cm. The antibacterial activity of the tube prepared in this Example was measured according to the above-described method.

### Example 5

10 wt % of copper sulfide synthesized as described in Example 1 was added to low-density polyethylene (LDPE; specific gravity: 0.92), and 1 wt % of zinc (Zn) particles were added thereto in order to reduce extrusion pressure. The mixture was subjected to a compounding process to thereby prepare chips. The prepared chips were extruded through an extruder at a temperature of 130° C. and an extrusion pressure of 0.1 (ΔP/h), thereby preparing a medical tube having a diameter of 1 cm and a length of 10 cm. The prepared tube was subjected to a two-step cooling process and an annealing process in order to improve the mechanical properties of the tube. The antibacterial activity of the tube prepared in this Example was measured according to the above-described method.

### Example 6

A medical tube having a diameter of 1 cm and a length of 10 cm was prepared in the same manner as described in Example 5, except that 5 wt % of copper sulfide and 0.2 wt % of manganese (Mn) were added to low-density polyethylene and that the extrusion pressure was 0.05 (ΔP/h) was used. The antibacterial activity of the tube prepared in this Example was measured according to the above-described method.

### Example 7

A medical tube having a diameter of 1 cm and a length of 10 cm was prepared in the same manner as described in Example 5, except that 20 wt % of copper sulfide and 0.6 wt % of iron (Fe) were added to high-density polyethylene (HDPE) and that the extrusion pressure was 0.2 (ΔP/h) was used. The antibacterial activity of the tube prepared in this Example was measured according to the above-described method.

### Example 8

A medical tube having a diameter of 1 cm and a length of 10 cm was prepared in the same manner as described in Example 5, except that 30 wt % of copper sulfide and 0.7 wt % of cobalt (Co) having an average particle diameter of 30 nm were added to polypropylene (PP) and that the extrusion pressure was 0.3 (ΔP/h) was used. The antibacterial activity of the tube prepared in this Example was measured according to the above-described method.

### Example 9

A medical tube having a diameter of 1 cm and a length of 10 cm was prepared in the same manner as described in Example 5, except that 40 wt % of copper sulfide and 2 wt % of chromium (Cr) were added to polyethylene terephthalate (PET) and that the extrusion pressure was 0.5 (ΔP/h) was used. The antibacterial activity of the tube prepared in this Example was measured according to the above-described method.

### Comparative Example 1

A medical tube made of low-density polyethylene (LDPE) and having a diameter of 1 cm and a length of 10 cm was prepared, and the antibacterial activity thereof was measured according to the above-described method.

### Comparative Example 2

A medical tube having a diameter of 1 cm and a length of 10 cm was prepared in the same manner as described in Example 5, except that 20 wt % of copper sulfide and 0.001 wt % of iron (Fe) were added to high-density polyethylene (HDPE) and that the extrusion pressure was 5 (ΔP/h). The antibacterial activity of the tube prepared in this Comparative Example was measured according to the above-described method.

### Comparative Example 3

A medical tube having a diameter of 1 cm and a length of 10 cm was prepared in the same manner as described in Example 5, except that 30 wt % of copper sulfide and 40 wt % of cobalt (Co) were added to polypropylene (PP) and that the extrusion pressure was 15 (ΔP/h) was used. The antibacterial activity of the tube prepared in this Comparative Example was measured according to the above-described method.

### Comparative Example 4

A medical tube having a diameter of 1 cm and a length of 10 cm was prepared in the same manner as described in Example 5, except that 40 wt % of copper sulfide and 2 wt % of aluminum (Al) were added to polyethylene terephthalate (PET) and that the extrusion pressure was 12 (ΔP/h) was used. The antibacterial activity of the tube prepared in this Comparative Example was measured according to the above-described method.

Table 1 below shows a comparison of the antibacterial activities (cells/mL) of the medical tubes prepared in Examples 1 to 6 and Comparative Examples 1 to 4. “Not measurable” in Table 1 means that the number of (Escherichia coli: ATCC 25922) cells was larger than 10¹⁰ which was not measurable.

TABLE 1

| | | | Electrically conductive | | | | |
|---|---|---|---|---|---|---|---|
| | | | particles | | | Medical tube | |
| | | | Copper | Metal | | Extru- | Antibac- |
| | | | sulfide | Kind | | sion | terial |
| | | Polymer | content | of | Content | pressure | activity |
| | | resin | (wt %) | metal | (wt %) | (Δ [symbol] /h) | (cells/mL) |
| Exam- | 1 | LDPE | 1 | / | / | / | 2.8 × 10⁶ |
| ples | 2 | LDPE | 10 | / | / | / | 5.8 × 10⁵ |
| | 3 | LDPE | 30 | / | / | / | 3.2 × 10⁴ |
| | 4 | LDPE | 0.1 | Zn | 1.5 | 0.07 | 4.0 × 10⁷ |
| | 5 | LDPE | 10 | Zn | 1 | 0.1 | 3.2 × 10⁶ |
| | 6 | LDPE | 5 | Mn | 0.2 | 0.05 | 6.5 × 10⁶ |
| | 7 | HDPE | 20 | Fe | 0.6 | 0.2 | 2.2 × 10⁵ |
| | 8 | PP | 30 | Co | 0.7 | 0.3 | 1.2 × 10⁵ |
| | 9 | PET | 40 | Cr | 2 | 0.5 | 1.3 × 10⁵ |
| Comp. | 1 | LDPE | / | / | / | / | Not |
| Exam- | | | | | | | mea- |
| ples | | | | | | | surable |
| | 2 | LDPE | 20 | Fe | 0.01 | 5 | 7.2 × 10⁵ |
| | 3 | PP | 30 | Co | 40 | 15 | 5.2 × 10¹⁰ |
| | 4 | PET | 40 | Al | 2 | 12 | 6.2 × 10¹⁰ |

[symbol]

indicates data missing or illegible when filed

Each of the coating solutions contained 1-30 wt % of copper sulfide. The tubes prepared in Examples 1 to 3 showed antibacterial activities of 2.8×10⁶ to 3.2×10⁴. However, the antibacterial activity of the tube of Comparative Example 1, which was not coated with copper sulfide, was very low such that it could not be measured. It could be seen that the antibacterial activity of the tubes coated with copper sulfide was higher than those of the tubes of Examples 4 to 9, which had copper sulfide dispersed by compounding. However, the time-dependent stability of the coating layer of copper sulfide can be lower than that of copper sulfide dispersed in the tube. The stability of the coating layer in some practical applications of the medical tube needs to be taken into consideration.

Regarding the medical tubes prepared by the compounding process, the medical tubes of Examples 4 to 9 had a copper sulfide content of 0.1-40 wt %. In addition, the metal particles were made of at least one selected from among chromium, manganese, iron, cobalt, nickel and zinc, and the concentration thereof was 0.1-2 wt % based on the total weight of the tube. The medical tubes prepared by the compounding process showed antibacterial activities of 1.2×10⁵ to 6.5×10⁶ cells/mL. In addition, the extrusion pressure was in the range of 0.05 to 0.5 (ΔP/h). However, the antibacterial activity of the tube of Comparative Example 1, which had no copper sulfide dispersed therein, was very low such that it could not be measured.

Comparative Example 2 did not satisfy an iron (Fe) metal particle concentration of 0.1-2 wt %, which was used in the Example of the present invention, and Comparative Example 3 did not satisfy a cobalt (Co) metal particle concentration of 0.1-2 wt %, which was used in the Example of the present invention. The tubes of Comparative Examples 2 and 3 showed antibacterial activities of 7.2×10⁵ cells/mL and 5.2×10¹⁰ cells/mL, respectively. Specifically, in Comparative Example 4 which used a metal particle concentration out of the metal particle concentration range used in the Examples of the present invention, the antibacterial activity of the tube was not significantly low, but the extrusion pressure was 15 (ΔP/h) which was not suitable for extrusion. In addition, in Comparative Example 3 which used a metal particle concentration out of the metal particle concentration range used in the Examples of the present invention, the extrusion pressure was 15 (ΔP/h), indicating that extrusion was impossible, and the antibacterial activity was also significantly low.

Comparative Example 4 is the case in which aluminum (Al) was added in place of the chromium, manganese, iron, nickel or zinc metal particles used in the present invention. In Comparative Example 4, the antibacterial activity was 6.2×10¹⁰ cells/mL, and the extrusion pressure was 12 (ΔP/h). Aluminum differs from transition metals belonging to group 4 of the periodic table. When aluminum was added, the antibacterial activity decreased, and the extrusion pressure also increased, resulting in a decrease in the efficiency with the tube was produced. Thus, the metal particles that are used in the present invention are preferably particles of a metal selected from among chromium, manganese, iron, cobalt, nickel and zinc, which are transition metal elements belonging to group 4 of the periodic table.

As described above, because the medical tube of the present invention, which comprises a copper-based compound, has a copper sulfide-containing compound coated thereon or dispersed therein, it is relatively inexpensive, is easy to process and is not toxic. In addition, the copper sulfide-containing compound that is used in the present invention has excellent antibacterial activity, and thus can be used to improve the antibacterial activity of medical tubes.

Although the preferred embodiments of the present invention have been described for illustrative purposes, the scope of the present invention is not limited to these embodiments, and those skilled in the art will appreciate that various modifications, additions and substitutions are possible, without departing from the scope and spirit of the invention as disclosed in the accompanying claims.

What is claimed is:

1. A medical tube comprising:
a tube formed of a polymer resin and having a predetermined shape and diameter; and
a copper-based compound coated on the surface of the tube or dispersed in the polymer resin of the tube,
wherein the compound has a chemical structure of CuₓM_{y}, wherein M is any one selected from groups 15 to 17 of the periodic table, and x/y is 0.5-1.5.

2. The medical tube of claim 1, wherein M is any one selected from among S, F and Cl.

3. The medical tube of claim 1, wherein the compound is copper sulfide.

4. The medical tube of claim 1, wherein the tube having the compound dispersed in the polymer resin comprises, based on the total weight of the tube, 0.1-5 wt % of metal particles of at least one selected from among chromium, manganese, iron, cobalt, nickel and zinc.

5. The medical tube of claim 4, wherein the average particle size of the metal particle is smaller than the average particle size of the compound.

6. The medical tube of claim 4, wherein coating of the copper-based compound on the surface of the tube is performed by any one method selected from among wet coating, vapor deposition, and plating.

7. The medical tube of claim 1, wherein a coating solution containing 0.01-1.0 wt % of colloidal transition metal particulates and 0.01-2.0 wt % of at least one emulsion selected from among water-soluble polyester, water-soluble urethane and water-soluble acryl is applied to the medical tube before the compound is coated on the medical tube.

8. The medical tube of claim 1, wherein the medical tube is any one selected from among tubes for infusion, enteral nutrition, peritoneal dialysis, transfusion, or transfer of urine into a urine collection bag, tubes for use in blood circuits for blood dialysis, blood circuits for artificial heart lung machines, or blood circuits for plasma exchange, tubes for mass transfer in the medical field, tubes for endoscopy, catheters and a plurality of tubes connected by a connector.

9. The medical tube of claim 7, wherein the tubes for mass transfer include a tube attached to a multiple blood bag, or a tube that is used to connect a suction unit to a catheter.

10. The medical tube of claim 8, wherein the catheter includes a urinary catheter, a gavage catheter or a suction catheter.

11. The medical tube of claim 1, wherein the medical tube is made of polyurethane resin or silicone resin.
